(19) United States
(12) Patent Application Publication
(10) Pub. No.: US 2008/0234756 A1
(43) Pub. Date: Sep. 25, 2008

(54) Pedicle Screw

(76) Inventors: John Sutcliffe, Good Easter (GB); Thomas Mack, Crailsheim (DE); Marcus Richter, Wiesbaden (DE); Nicholas Willmann, Ulm (DE)

Correspondence Address: COLLARD & ROE, P.C., 1077 NORTHERN BOULEVARD, ROSLYN, NY, 11576, US

(21) Appl. No.: 10/592,611

(22) PCT Filed: Dec. 1, 2004
(86) PCT No.: PCT/DE2004/002644

§ 371 Date: May 25, 2007

Related U.S. Application Data
(60) Provisional application No. 60/472,578, filed on May 22, 2003.
(60) Provisional application No. 60/527,060, filed on Dec. 4, 2003.

(30) Foreign Application Priority Data
Nov. 19, 2002 (FR) 02/14454
Mar. 10, 2004 (DE) 10 2004 011 6121.

(51) Int. Cl. A61B 17/04

(52) U.S. Cl. 606/308; 606/301; 606/305

(57) ABSTRACT

The invention relates to a pedicle screw for implants for the correction and stabilization of the spinal column, having a head part (4) disposed on the one axial end of a threaded shaft (2), to which part a shackle part (3) can be connected, which part has a shackle accommodation (5) for a rod (6) that can be fixed in place on the head part (4). A threaded bore (8) is formed in the shackle part (3), to accommodate a thread that is assigned to the head part (4).

The invention relates to a pedicle screw for implants for the correction and stabilization of the spinal column, having a head part disposed on the one axial end of a threaded shaft, to which part a shackle part can be connected, which part has a shackle accommodation for a rod that can be fixed in place on the head part.

Such pedicle screws are known from practice, and serve for being screwed into the vertebrae or a spinal column, in order to make it possible for the spinal column to perform its function in case of traumatic or degenerative diseases, in that at least one rod that bridges several vertebrae is passed along the spinal column and anchored to it. For a precise orientation of the rod, it is necessary to be able to adapt the position of the pedicle screw to the rod, at least at the contact point, taking into consideration the anatomically correct position of the pedicle screw within the vertebra. In practice, therefore, very many pedicle screws are kept on hand for an operation, making it possible to take into consideration the different sizes of the vertebrae, as a function of their position in the cervical spinal column, the thoracic spinal column, or the lumbar spinal column, and this increases the costs in production, warehousing, and stock kept on hand for an operation.

The invention is therefore based on the task of further developing a pedicle screw of the type stated initially, in such a manner that these disadvantages can be avoided.

This task is accomplished, according to the invention, in the case of a pedicle screw of the type stated initially, in that a threaded bore is formed in the shackle part, to accommodate a thread that is assigned to the head part.

The advantage connected with this embodiment is that the threaded shaft and its shackle part are present as separate components, which can be made available in different variants, so that a very great number of different pedicle screws can be compiled by combining these variants. Furthermore, there is also the advantage that the threaded shaft can be introduced into the vertebrae without hindrance by the shackle part, at first, and its correct position can be checked, and that the shackle part with the threaded bore can be screwed onto the threaded shaft afterwards.

In this connection, it is preferred, within the scope of the invention, if the head part is formed as a spherical head and the thread is formed on the outside of a thread cage, the cage interior of which forms a ball socket for the ball head. By means of this embodiment, the possibility of making available a flexible connection between the threaded shaft and the rod by way of the shackle part is created, so that precise positioning of the rod is possible, by way of the polyaxial alignment possibility of the shackle part with its shackle accommodation, relative to the axis of the threaded shaft.

In order to be able to screw the threaded shaft into the vertebra also independent of the shackle part, the embodiment is designed in such a manner that a non-circular, preferably multi-angular tool accommodation for a tool that exerts a torque is provided in the free end of the head part, coaxial to the axis of the threaded shaft.

It is also advantageous if a coaxial guide accommodation is formed in the threaded shaft, from which at least one side channel leads to the circumference surface of the threaded shaft. This guide accommodation offers the possibility of pressing a binder, such as bone cement or other adhesives, for example, into the bone opening once the pedicle screw has been placed into the vertebra, and thereby of allowing a better seat of the pedicle screw in the vertebra, on which stress can be placed at an early point in time and over a long term. It must be taken into consideration, in this connection, that the mobility of the shackle part relative to the threaded shaft is not impaired when this happens, since the shackle part can still be separate from the threaded shaft, and-therefore the application of the binder is not impaired, and a correspondingly clean method of working is possible, which also allows removal of excess binder before the shackle part is screwed on. Rapid and uniform distribution of the binder is achieved in that the side channel is provided multiple times, uniformly distributed over the circumference of the threaded shaft, or multiple times, axially staggered.

It is furthermore preferred if the side channel opens into the circumference surface of the threaded shaft between the thread channels, since in this way, the thread cutting edges remain uninfluenced, and screwing the threaded shaft in is not impaired.

If the guide accommodation extends over the entire axial length of the threaded shaft, there is the possibility of filling a drilled channel, which is placed before the threaded shaft is screwed in, completely with binder. Furthermore, there is the very great advantage that this pedicle screw, which is made into a cannula by means of the continuous guide accommodation, can also be used in a “percutaneous” application, within the scope of minimally invasive surgery, in that a guide wire is first introduced into the operating field, onto which the threaded shaft with the guide accommodation is threaded, in order to be guided to the correct position along this guide wire.

It is practical if the arrangement is made in such a manner that the threaded bore is disposed in the shackle base of the shackle part, and that a ring can be set onto the head part through the shackle accommodation. An improved contact surface for the rod is made available by means of this ring, and furthermore, the possibility is created of pressing the ring onto the head part by means of applying external pressure, and thereby terminating the mobility of the head part relative to the shackle part.

In order to permanently fix the rod in position in the shackle accommodation, a lid is connected with the first shank of the shackle part, by way of an articulation. In this connection, it is preferred, within the scope of the invention, if a shank tongue projects from the second shank of the shackle part, radially towards the outside, towards the side that faces away from the first shank, in which tongue a tongue threaded bore for accommodating a locking screw that passes through an opening in the lid is formed. With this embodiment, there is the advantage that the axis of the tongue threaded bore does not agree identically with the axis of the threaded shaft or the axis of the threaded bore, but rather is laterally offset. This prevents a torque from being exerted on the threaded shaft when the locking screw is being screwed in, particularly when it reaches its firm seat, which torque could loosen the seat of the threaded shaft in the vertebra or damage the binder that has been pressed in through the guide accommodation, as it is curing.

During an operation, the necessity often arises of performing precise adjustments with regard to the position of the vertebra, particularly distractions or compressions, after the position and orientation of the pedicle screw have been fixed in place. However, as long as the position of the threaded shaft relative to the head part has not been fixed in place so that it can withstand stress, in this connection, a force exerted on the vertebrae often does not have a distracting or compressing effect, and instead results in tipping movements. Therefore, in order to be able to fix the orientation of the threaded shaft relative to the shackle part in place, permanently and able to withstand stress, and thereby to allow a rotation of the rod as well as a translational displacement relative to the shackle part, a punch is disposed between the lid and the head part, to clamp the head part relative to the shackle part when final closing of the lid occurs. By means of clamping the head part in the shackle part, the relative position of the threaded shaft relative to the shackle part is fixed in place and secured, while the rod can still be adjusted relative to the shackle part. In this way, precise, also iterative orientation of the vertebrae is possible during the operation.

The final fixation of the shackle part relative to the rod is achieved in simple manner, in that a screw accommodation is formed in the lid, to accommodate a fixation screw that serves to clamp the rod in place.

It is practical if the punch is formed by means of a ring shank that is formed on the ring, so that the ring provided to clamp the head part is impacted not only by the rod, but also by the punch that is formed in one piece with the ring. This also reduces the number of components required to complete the pedicle screw.

In order to be able to achieve a uniform introduction of force into the head part, there is the possibility that the ring shank is provided twice, on opposite sides of the ring, to surround the rod on both sides.

For a precise transfer of force from the lid onto the punch, it is furthermore possible that a pressure strip is formed on the shackle, on the side that faces the head part, to impact the punch.

In order to make a great force available to impact the punch, it is advantageous if the contact surface for the punch that is formed on the shackle part is disposed at a distance from the articulation, in other words an intensification of force can be achieved by way of a lever arm formed by the lid, and an additional parameter is available, by means of the length of the lever arm, to determine the clamping force.

In the following, the invention will be explained in greater detail using exemplary embodiments shown in the drawing; this shows:

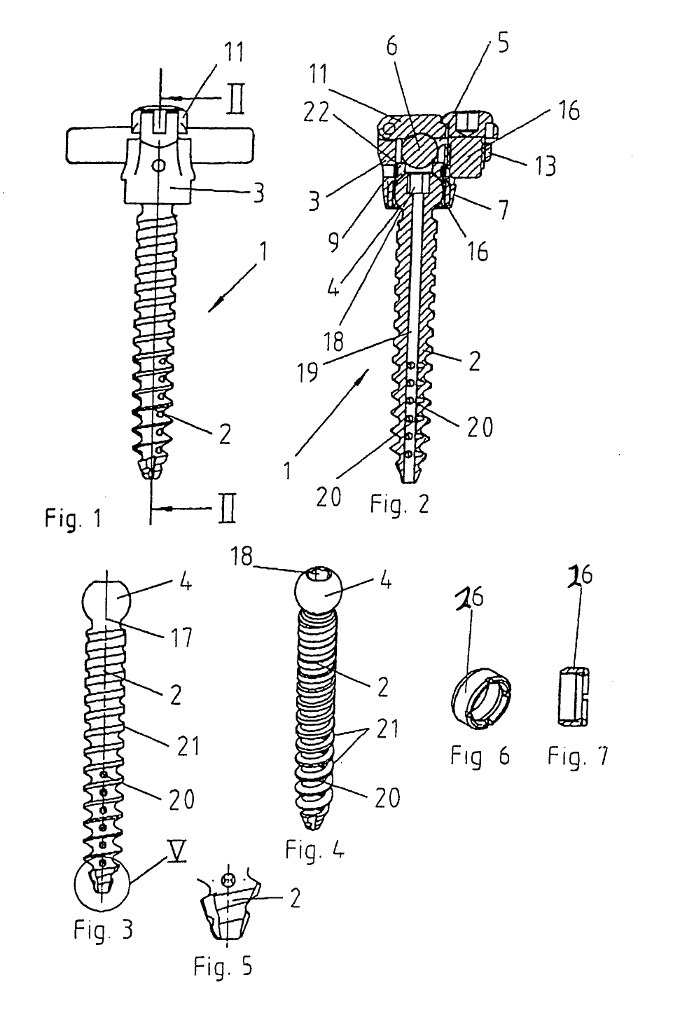

FIG. 1 a side view of the pedicle screw according to the invention,

FIG. 2 the section II-II from FIG. 1,

FIG. 3 in a side view, the isolated threaded shaft with the head part formed as a spherical head,

FIG. 4 a perspective representation of the threaded shaft from FIG. 3,

FIG. 5 the detail V from FIG. 3,

FIG. 6 a perspective representation of the thread cage,

FIG. 7 a longitudinal section through the thread cage from FIG. 6,

FIG. 8 a perspective representation of the ring,

FIG. 9 a longitudinal section through the ring from FIG. 8,

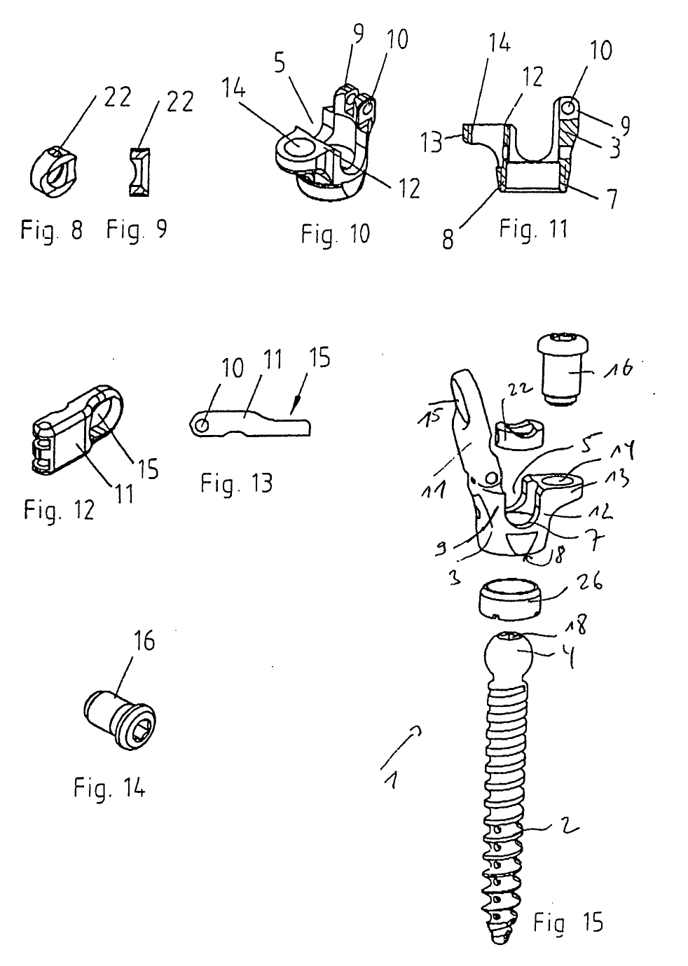

FIG. 10 a perspective representation of the shackle part,

FIG. 11 a longitudinal section through the shackle part from FIG. 10,

FIG. 12 a perspective representation of the lid,

FIG. 13 a side view of the lid from FIG. 12,

FIG. 14 a perspective representation of the locking screw,

FIG. 15 an exploded representation of the pedicle screw,

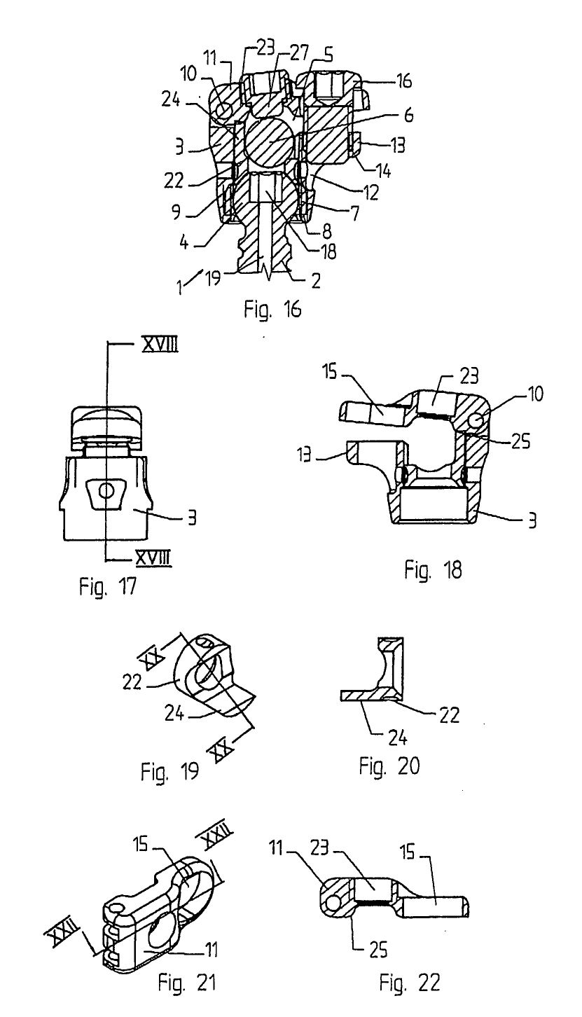

FIG. 16 a longitudinal section through the upper region of a further embodiment,

FIG. 17 a front view of the shackle part with the lid of the further embodiment,

FIG. 18 the section XVIII-XVIII from FIG. 17,

FIG. 19 a perspective representation of the modified ring of the further embodiment,

FIG. 20 the section XX-XX from FIG. 19,

FIG. 21 a perspective representation of the lid of the modified embodiment, and

FIG. 22 the section XXII-XXII from FIG. 21.

In the drawing, a pedicle screw 1 for implants is shown, which consists of a threaded shaft 2 and a shackle part 3. On the one axial end of the threaded shaft 2, a head part 4 is formed, which is shaped as a spherical head. The shackle part 3 has a shackle accommodation 5 for a rod 6 that can be fixed in place on the head part 4, whereby a threaded bore 8 is formed in the shackle base 7 of the shackle part 3. Furthermore, the shackle part 3 has two shanks, whereby a lid 11 is connected to the first shank 9, by way of an articulation 10, and a shank tongue 13 projects away from the second shank 12, radially towards the outside, to the side that faces away from the first shank 9, into which tongue a tongue threaded bore 14 for accommodating a locking screw 16 that passes through the lid 11 in an opening 15 is formed.

The head part 4 has a thread assigned to it, namely on the outside of the thread cage 26, the interior of which cage forms a ball socket for the spherical head of the head part 4, whereby the interior of the cage can be structured as an inside cone or a partial sphere.

A non-circular tool accommodation 18 is made in the free end of the head part 4, coaxially to the axis 17 of the threaded shaft 2, by means of which a torque can be exerted on the threaded shaft 2, by means of a tool, and thereby the threaded shaft 2 can be screwed into a vertebra. The threaded shaft 2 furthermore has a coaxial guide accommodation 19, from which several axially staggered side channels 20 lead to the circumference surface of the threaded shaft 2 and open between the thread channels 21 there; in the exemplary embodiment shown, the side channels are uniformly distributed over the circumference. The guide accommodation 19 extends over the entire axial length of the threaded shaft 2, which is provided with a self-tapping thread (FIG. 5) at the end opposite the head part 4.

In FIGS. 16 to 22, an embodiment is shown in which a punch 28 is disposed between the lid 11 and the head part 4, to clamp the head part 4 in place relative to the shackle part 3, during final closing of the lid 11, whereby furthermore, a screw accommodation 23 is formed in the lid 11, to accommodate a fixation screw 27 that serves to clamp the rod 6 in place. In this connection, the punch 28 is implemented by means of a ring shank 24 formed on the ring 22, which shank can also be provided twice, according to an embodiment not shown in the drawing, on opposite sides of the ring 22, to surround the rod 6 on both sides.

It is evident, particularly from FIG. 22, that a pressure strip 25 is formed on the lid 11, on the side facing the head part 4, to impact the punch 28. The contact surface formed on the lid 11, in FIG. 22 the pressure strip 25, is disposed at a distance from the articulation 10, for the punch 28.

By means of the configuration of the pedicle screw 1 according to the invention, the possibility arises of first screwing the threaded shaft 2 into the vertebra and permanently anchoring it there by means of bone cement, which is pressed in through the guide accommodation 19 and the side channels 20. Subsequently, the shackle part 3 is then attached to the threaded shaft 2, in that the shackle part 3 is screwed onto the thread cage 16 with the threaded bore 8. While the lid 11 of the shackle part 3 is open, the rod 6 is then placed onto a ring 12, and as the lid 11 is being closed, the rod is pressed onto the ring 22 by the lid, and therefore onto the head part 4 of the threaded shaft 2, in order to thereby terminate the movement possibility of the shackle part 3 relative to the threaded shaft 2. Locking of the lid 11 and thereby of the rod 6 takes place with the locking screw 16, in the case of the first embodiment according to FIGS. 1 to 15.

The embodiment from FIGS. 16 to 22 offers increased variability, since at first, the lid 11 can be closed by means of the locking screw 16, thereby also terminating the rotation possibility of the head part 4 relative to the shackle part 3. The shackle part 3 itself, however, can still rotate relative to the rod 6, in other words it can be rotated about the axis of the latter and also be displaced parallel to this longitudinal axis. These movement possibilities are eliminated by means of the fixation screw 27, when the pedicle screw 1 has assumed the desired position relative to the vertebra. It must be noted that the fixation screw 27 acts directly on the rod 6, which in turn reinforces the clamping of the head part 4 by way of the ring 22.

### REFERENCE SYMBOL LIST

1 pedicle screw
2 threaded shaft
3 shackle part
4 head part
5 shackle accommodation
6 rod
7 shackle base
8 threaded bore
9 first shank
10 articulation
11 lid
12 second shank
13 shank tongue
14 tongue threaded bore
15 opening
16 locking screw
17 axis of the threaded shaft
18 tool accommodation
19 guide accommodation
20 side channels
21 thread channels
22 ring
23 screw accommodation
24 ring shank
25 pressure strip
26 thread cage
27 fixation screw
28 punch

1. Pedicle screw for implants for the correction and stabilization of the spinal column, having a head part (4) disposed on the one axial end of a threaded shaft (2), to which part a shackle part (3) can be connected, which part has a shackle accommodation (5) for a rod (6) that can be fixed in place on the head part (4), characterized in that a threaded bore (8) is formed in the shackle part (3), to accommodate a thread that is assigned to the head part (4).

2. Pedicle screw according to claim 1, characterized in that the head part (4) is formed as a spherical head and the thread is formed on the outside of a thread cage (26), the cage interior of which forms a ball socket for the ball head.

3. Pedicle screw according to claim 1, characterized in that a non-circular, preferably multi-axial tool accommodation (18) is made in the free end of the head part (4), coaxially to the axis (17) of the threaded shaft (2), for a tool that serves to exert a torque.

4. Pedicle screw according to one of claim 1, characterized in that a coaxial guide accommodation (19) is formed in the threaded shaft (2), from which at least one side channel (20) leads to the circumference surface of the threaded shaft (2).

5. Pedicle screw according to claim 4, characterized in that the side channel (20) is provided multiple times, uniformly distributed over the circumference of the threaded shaft (2).

6. Pedicle screw according to claim 4, characterized in that the side channel (20) is provided multiple times, axially staggered.

7. Pedicle screw according to claim 4, characterized in that the side channel (20) opens into the circumference surface of the threaded shaft (2) between the thread channels (21).

8. Pedicle screw according to claim 4, characterized in that the guide accommodation (19) extends over the entire axial length of the threaded shaft (2).

9. Pedicle screw according to claim 1, characterized in that the threaded bore (8) is disposed in the shackle base (7) of the shackle part (3), and that a ring (22) can be set onto the head part (4) through the shackle accommodation (5).

10. Pedicle screw according to claim 1, characterized in that a lid (11) is connected with the first shank (9) of the shackle part (3), by way of an articulation (10).

11. Pedicle screw according to claim 10, characterized in that a shank tongue (13) projects from the second shank (12) of the shackle part (3), radially towards the outside, to the side facing away from the first shank (9), in which tongue a tongue threaded bore (14) for accommodating a locking screw (16) that passes through an opening (15) in the lid (11) is formed.

12. Pedicle screw according to claim 10, characterized in that a punch (28) is disposed between the lid (11) and the head part (4), to clamp the head part (4) relative to the shackle part when final closing of the lid (11) occurs.

13. Pedicle screw according to claim 12, characterized in that a screw accommodation (23) is formed in the lid (11), to accommodate a fixation screw (27) that serves to clamp the rod (6) in place.

14. Pedicle screw according to claim 12, characterized in that the punch (28) is formed by means of a ring shank (24) that is formed on the ring (22).

15. Pedicle screw according to claim 14, characterized in that the ring shank (24) is provided twice, on opposite sides of the ring (22), to surround the rod (6) on both sides.

16. Pedicle screw according to claim 12, characterized in that a pressure strip (25) is formed on the lid (11), on the side that faces the head part (4), to impact the punch (28).

17. Pedicle screw according to claim 12, characterized in that the contact surface for the punch (28) that is formed on the lid (11) is disposed at a distance from the articulation (10).
